(19) United States
(12) Patent Application Publication
(10) Pub. No.: US 2006/0172328 A1
(43) Pub. Date: Aug. 3, 2006

(54) Methods and compositions for correcting misincorporation in a nucleic acid synthesis reaction

(76) Inventor: Philip R. Buzby, Brockton, MA (US)

Correspondence Address: PROSKAUER ROSE LLP, ONE INTERNATIONAL PLACE 14TH FL, BOSTON, MA, 02110, US

(21) Appl. No.: 11/326,121

(22) Filed: Jan. 5, 2006

Related U.S. Application Data
(60) Provisional application No. 60/641,388, filed on Jan. 5, 2005.

(51) Int. Cl. C12Q 1/68; C12P 19/34

(52) U.S. Cl. 435/6; 435/91.2

(57) ABSTRACT

The invention provides methods for correcting misincorporation of a nucleotide in a primer during a sequencing-by-synthesis reaction by using both a polymerase substantially lacking in exonuclease activity and an enzyme, preferably a polymerase, having exonuclease activity.

### REFERENCE TO RELATED APPLICATIONS

This application claims the benefit of and priority to U.S. provisional application No. 60/641,388, filed Jan. 5, 2005, the entirety of which is hereby incorporated by reference.

### TECHNICAL FIELD OF THE INVENTION

The invention generally relates to methods for correcting misincorporation of nucleotides in a nucleic acid synthesis reaction, and more particularly to methods for sequencing a nucleic acid using both a polymerase substantially lacking in exonuclease activity and an enzyme with exonuclease activity.

### BACKGROUND OF THE INVENTION

Completion of the human genome has paved the way for important insights into biologic structure and function. Knowledge of the human genome has given rise to inquiry into individual differences, as well as differences within an individual, as the basis for differences in biological function and dysfunction. For example, single nucleotide differences between individuals, called single nucleotide polymorphisms (SNPs), are responsible for dramatic phenotypic differences. Those differences can be outward expressions of phenotype or can involve the likelihood that an individual will get a specific disease or how that individual will respond to treatment. Moreover, subtle genomic changes have been shown to be responsible for the manifestation of genetic diseases, such as cancer. A true understanding of the complexities in either normal or abnormal function will require large amounts of specific sequence information.

An understanding of cancer also requires an understanding of genomic sequence complexity. Cancer is a disease that is rooted in heterogeneous genomic instability. Most cancers develop from a series of genomic changes, some subtle and some significant, that occur in a small subpopulation of cells. Knowledge of the sequence variations that lead to cancer will lead to an understanding of the etiology of the disease, as well as ways to treat and prevent it. An essential first step in understanding genomic complexity is the ability to perform high-resolution sequencing.

Various approaches to nucleic acid sequencing exist. One conventional way to do bulk sequencing is by chain termination and gel separation, essentially as described by Sanger et al., Proc. Natl. Acad. Sci., 74(12): 5463-67 (1977). That method relies on the generation of a mixed population of nucleic acid fragments representing terminations at each base in a sequence. The fragments are then run on an electrophoretic gel and the sequence is revealed by the order of fragments in the gel. Another conventional bulk sequencing method relies on chemical degradation of nucleic acid fragments. See, Maxam et al., Proc. Natl. Acad. Sci., 74: 560-64 (1977). Finally, methods have been developed based upon sequencing by hybridization. See, e.g., Drmanac, et al., Nature Biotech., 16: 54-58 (1998).

Bulk sequencing techniques are not useful for the identification of subtle or rare nucleotide changes due to the many cloning, amplification and electrophoresis steps that complicate the process of gaining useful information regarding individual nucleotides. The ability to sequence and gain information from single molecules obtained from an individual patient is the next milestone for genomic sequencing. As such, research has evolved toward methods for rapid sequencing, such as single molecule sequencing technologies.

Single molecule sequencing techniques allow the evaluation of individual nucleic acid molecules in order to identify changes and/or differences affecting genomic function. In single molecule techniques, individual, optically-resolvable nucleic acid fragments are attached to a solid support, and sequencing is conducted on the individual strands. Sequencing events are detected and correlated to the individual strands. See Braslavsky et al., Proc. Natl. Acad. Sci., 100: 3960-64 (2003), incorporated by reference herein. Because single molecule techniques do not rely on ensemble averaging as do bulk techniques, errors due to misincorporation can have a significant deleterious effect on the sequencing results. The misincorporation of nucleotides, i.e., the incorporation of a nucleotide that incorrectly paired with a corresponding template nucleotide, during the synthesis of primer extension products limits the length of the sequence that can be determined. The presence of misincorporated nucleotides may result in prematurely terminated strand synthesis, reducing the number of template strands for future rounds of synthesis, and thus reducing the efficiency of sequencing. The frequency of misincorporation is a measure of the accuracy or fidelity of the nucleic acid polymerase that is used in the synthesis reaction. The fidelity of the polymerization is maintained by both the polymerase activity and the 3′→5′ exonuclease activity of the polymerase. In the presence of all four dNTPs, misincorporation frequencies by DNA polymerases possessing 3′→5′ exonuclease activity are as low as one error in 10⁶ to 10⁸ nucleotides incorporated, while in the absence of 3′→5′ exonuclease activity, DNA polymerase error rates are typically about one error in 10⁴ to 10⁶. See Echols and Goodman (1991, Annu. Rev. Biochem 60:477-511); and Goodman et al. (1993, Crit. Rev. Biochem. Molec. Biol. 28:83-126); and Loeb and Kunkel (1982, Annu. Rev. Biochem. 52:429-457). Although exonuclease activity increases the fidelity of a DNA polymerase, the use of an exo(+) polymerase often results in significantly decreased product yields because, in the absence of a correct nucleotide complementary to the next template base, the exonuclease also will remove correctly-paired nucleotides successively. There is, therefore, a need in the art for improved methods for reducing or eliminating misincorporations in nucleic acid sequencing, especially single molecule sequencing.

### SUMMARY OF THE INVENTION

The invention addresses the problem of misincorporations in nucleic acid sequencing-by-synthesis reactions—especially single molecule sequencing. A misincorporation event in a bulk sequencing reaction has a negligible effect on the overall result due to the numerous copies of predominant sequence that are available in bulk techniques. However, in single molecule sequencing, a sequencing error in any individual strand is significant. Moreover, misincorporated bases typically inhibit or terminate further chain extension—which also is a significant problem in single molecule techniques. Methods of the invention are equally applicable in bulk and single molecule techniques and have advantages for both.

The invention reduces the deleterious effects of misincorporations by providing limited proofreading during primer extension. According to the invention, a sequencing reaction is conducted in which a template nucleic acid to be sequenced is hybridized to a primer for template-dependent chain elongation. The template/primer duplex is exposed to a first polymerase lacking exonuclease activity; a second enzyme, preferably a polymerase, having exonuclease activity; and one or more nucleotide triphosphates (i.e., adenosine triphosphate, guanidine triphosphate, cytidine triphosphate, uridine triphosphate and/or thymidine triphosphate) for incorporation into the primer under conditions that allow sequential incorporation of complementary bases into the primer. The duplex can be exposed to the nucleotides simultaneously or sequentially one at a time. The second polymerase is preferably present in an amount sufficient to remove most or all of any nucleotides that are misincorporated (i.e., mismatched under standard Watson-Crick base pairing) into the primer, but insufficient to cause substantial removal of correctly-incorporated nucleotides. As a result, misincorporated nucleotides are removed, making the strand that bore the misincorporation available for incorporation of the proper (i.e., complementary) nucleotide. Methods of the invention correct some or all of the misincorporation errors that result in erroneous base calling and/or chain termination in single molecule sequencing reactions. A sequence of the template is compiled based upon the sequence of nucleotide incorporated into the primer.

According to the invention, first and second polymerase preferably are introduced into the sequencing reaction together, but can also be introduced in series. In a preferred embodiment, the amount of first polymerase is in excess relative the amount of second polymerase in order to drive the majority of strands toward incorporation of the proper nucleotide. The amount of second polymerase (exo(+)), i.e., proofreading DNA polymerase, used in the reaction is determined based upon the misincorporation rate. That rate is dependent on the species of nucleotide (i.e., dATP, dGTP, dCTP, dUTP or dTTP) being incorporated and on the nucleotide immediately 5′ on the primer (i.e., the nucleotide adjacent the incorporation site). Thus, methods of the invention are amenable to fine tuning with respect to the relative amounts of exo(−) and exo(+) polymerase as described herein. In general, however, the amount of exo(+) polymerase is chosen empirically to achieve a predetermined desired amount of proofreading of misincorporated bases.

Single molecule sequencing methods of the invention preferably comprise template/primer duplex attached to a surface. Individual nucleotides added to the surface comprise a detectable label—preferably a fluorescent label. Each species of nucleotides can comprise a different label, or they can comprise the same label. Each duplex is individually optically resolvable in order to facilitate single molecule sequence discrimination. The choice of a surface for attachment of duplex depends upon the detection method employed. Preferred surfaces for methods of the invention are epoxide surfaces and polyelectrolyte multilayer surfaces, such as those described in Braslavsky, et al., supra. Surfaces preferably are deposited on a substrate that is amenable to optical detection of the surface chemistry, such as glass or silica. The precise surface and substrate used in methods of the invention is immaterial to the functioning of the invention as long as the misincorporation proofreading function described above is enabled.

In a preferred embodiment, template-dependent nucleic acid sequencing is conducted in the presence of a first polymerase having reduced exonuclease activity and a second polymerase that retains exonuclease activity, such that the first polymerase is present in excess relative to the second polymerase. Labeled deoxynucleotide triphosphates are added in the sequencing reaction under conditions that allow the incorporation of a dNTP that is complementary to a template nucleotide that is adjacent to the 3′ terminus of the primer. The second polymerase is present in an amount sufficient to remove incorrectly-incorporated nucleotides that have been added to the primer but insufficient to remove a substantial amount of correctly-incorporated nucleotide. The second polymerase can be present in any amount relative to the first polymerase, though preferred amounts include about 1% or less, less than about 0.5%, and less than about 0.1% than the amount of the first polymerase. The second polymerase can be present at greater relative amounts, such as about the same or an equimolar amount. In the latter case, the second polymerase can be exposed to the primer/target duplex for a shorter period of time than the first polymerase.

In one feature, methods of the invention take into account that different nucleotides incorporate in an extending primer at different rates and that misincorporation rates similarly vary with the type of nucleotide being incorporated. For example, as shown in FIG. 1, guanidine nucleotides have a significantly higher misincorporation rate than do thymidine nucleotides, for example. Accordingly, the amount of exo(+) polymerase used in methods of the invention can be varied with the identity of the correct nucleotide sought to be incorporated.

In another feature of the invention, the second exo(+) polymerase is exposed to the reaction mixture in a separate step but for a time that allows proofreading of misincorporation events without significant or substantial removal of correctly incorporated bases. For example, in one embodiment, an exo(−) polymerase is exposed to duplex and free nucleotides for a period from about 5 times to about 100 times as long as the period during which exo(+) polymerase is exposed to the mixture.

Polymerases useful in the invention include any polymerizing agent capable of catalyzing a template-dependent addition of a nucleotide or nucleotide analog to a primer. Depending on the characteristics of the target nucleic acid, a DNA polymerase, an RNA polymerase, a reverse transcriptase, or a mutant or altered form of any of the foregoing can be used. According to one aspect of the invention, a thermophilic polymerase is used, such as ThermSequenase®, 9°N^{TM}, Therminator™, Taq, Tne, Tma, Pfu, Tfl, Tth, Tli, Stoffel fragment, Vent™ and Deep Vent™ DNA polymerase. In one embodiment, the invention provides for the primer/target nucleic acid complex to be exposed to the polymerase and nucleotide at a temperature between about 30° and about 80′Celsius. A preferred polymerase is a Klenow fragment DNA polymerase having reduced 3′→5′ exonuclease activity.

### BRIEF DESCRIPTION OF THE DRAWING

FIG. 1 shows the difference in misincorporation rates of guanidine nucleotides versus other nucleotides, including thymidine nucleotides.

### DETAILED DESCRIPTION

The invention provides methods and compositions for correcting misincorporation of nucleotides in a nucleic acid sequencing-by-synthesis reaction. While applicable to bulk sequencing methods, the invention is particularly useful in connection with single molecule sequencing methods. According to the invention, the fidelity of DNA synthesis is improved by adding small amount of 3′→5′ exonuclease activity to a nucleic acid synthesis reaction. The proofreading activity improves the yield of sequencing information by removing misincorporated nucleotides and permitting complete strand synthesis by the predominant polymerase activity.

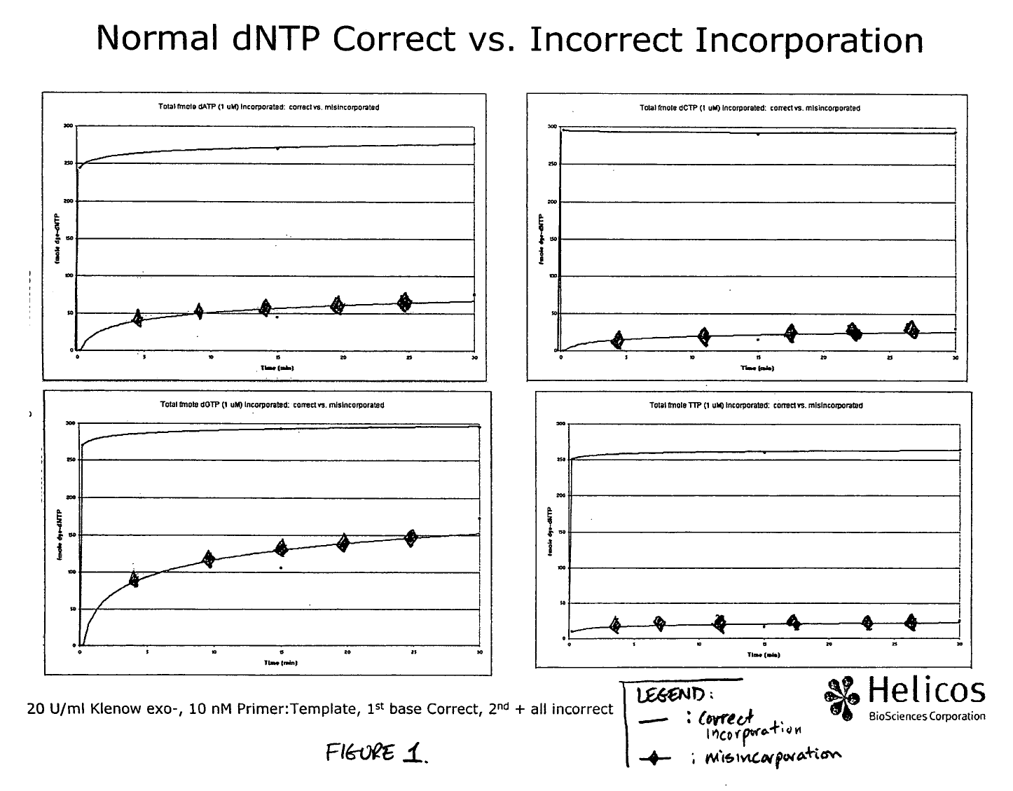

According to the invention, a sequencing reaction is conducted in which a template nucleic acid to be sequenced is hybridized to a primer for template-dependent chain elongation. A polymerization reaction is performed in the presence of one or more nucleotides or nucleotide analogs and a first polymerase substantially lacking exonuclease activity. The reaction is performed under conditions that allow template-dependant incorporation of nucleotides into the primer. The template/primer duplex also is exposed to a second enzyme having 3′→45′ exonuclease activity, either simultaneously with or subsequently to the first polymerase. In a preferred embodiment, the second enzyme is a polymerase having exonuclease proofreading activity. The second polymerase is preferably present in an amount sufficient to remove most or all of any nucleotides that are misincorporated into the primer, but insufficient to cause substantial removal of correctly-incorporated nucleotides. According to one aspect of the invention, the amount of second polymerase (exo(+)) used in the reaction is determined based upon the misincorporation rate. That rate can, among other things, be dependent on the species of nucleotide being incorporated or on the nucleotide immediately 5′ on the primer (i.e., the nucleotide adjacent the incorporation site). For example, as shown in FIG. 1, guanidine nucleotides have a significantly higher misincorporation rate than do thymidine nucleotides, for example. Accordingly, the amount of exo(+) polymerase used in methods of the invention can be varied with the identity of the correct nucleotide sought to be incorporated. A sequence of the template is compiled based upon the sequence of nucleotide incorporated into the primer.

In a preferred embodiment, template-dependent nucleic acid sequencing is conducted in the presence of a first polymerase having reduced exonuclease activity and a second polymerase that retains exonuclease activity, such that the first polymerase is present in excess relative to the second polymerase. The second polymerase can be present in any amount relative to the first polymerase, though preferred amounts include about 1% or less, less than about 0.5%, and less than about 0.1% than the amount of the first polymerase.

In another aspect of the invention, the second polymerase can be present at an amount greater than 1% of the first polymerase, such as about the same amount, but the second exo(+) polymerase is exposed to the reaction mixture in a separate step but for a time that allows proofreading of misincorporation events without significant or substantial removal of correctly incorporated bases. For example, in one embodiment, an exo(−) polymerase is exposed to duplex and free nucleotides for a period from about 5 times to about 100 times as long as the period during which exo(+) polymerase is exposed to the mixture.

Single molecule sequencing methods of the invention preferably comprise template/primer duplex attached to a surface such that each duplex is individually optically resolvable. Individual nucleotides added to the surface comprise a detectable label—preferably a fluorescent label. Each species of nucleotides can comprise a different label, or they can comprise the same label. The species of nucleotides can be present in the reaction mixture either individually, all at one, or in any combination of two or three.

Certain non-limiting aspects of the invention are further described below in terms of general considerations and examples.

I. General Considerations

A. Nucleic Acid Polymerases

Nucleic acid polymerases generally useful in the invention include DNA polymerases, RNA polymerases, reverse transcriptases, and mutant or altered forms of any of the foregoing. DNA polymerases and their properties are described in detail in, among other places, DNA Replication 2nd edition, Komberg and Baker, W. H. Freeman, New York, N.Y. (1991). Polymerase and exonuclease activity vary with each polymerase enzyme. However, any polymerase can be modified to add or eliminate exonuclease activity, or to increase or decrease polymerase or exonuclease activity. Assays for DNA polymerase activity and 3→5′ exonuclease activity can be found in DNA Replication 2nd Ed., Komberg and Baker, supra; Enzymes, Dixon and Webb, Academic Press, San Diego, Calif. (1979), as well as other publications available to the person of ordinary skill in the art.

Known conventional DNA polymerases useful in the invention include, but are not limited to, Pyrococcus furiosus (Pfu) DNA polymerase (Lundberg et al., 1991, Gene, 108: 1, Stratagene), Pyrococcus woesei (Pwo) DNA polymerase (Hinnisdaels et al., 1996, Biotechniques, 20:186-8, Boehringer Mannheim), Thermus thermophilus (Tth) DNA polymerase (Myers and Gelfand 1991, Biochemistry 30:7661), Bacillus stearothermophilus DNA polymerase (Stenesh and McGowan, 1977, Biochim Biophys Acta 475:32), Thermococcus litoralis (Tli) DNA polymerase (also referred to as Vent™ DNA polymerase, Cariello et al., 1991, Polynucleotides Res, 19: 4193, New England Biolabs), 9°Nm™ DNA polymerase (New England Biolabs), Stoffel fragment, ThermoSequenase® (Amersham Pharmacia Biotech UK), Therminator™ (New England Biolabs), Thermotoga maritima (Tma) DNA polymerase (Diaz and Sabino, 1998 Braz J Med. Res, 31:1239), Thermus aquaticus (Taq) DNA polymerase (Chien et al., 1976, J. Bacteoriol, 127: 1550), DNA polymerase, Pyrococcus kodakaraensis KOD DNA polymerase (Takagi et al., 1997, Appl. Environ. Microbiol. 63:4504), JDF-3 DNA polymerase (from thermococcus sp. JDF-3, Patent application WO 0132887), Pyrococcus GB-D (PGB-D) DNA polymerase (also referred as Deep Vent™ DNA polymerase, Juncosa-Ginesta et al., 1994, Biotechniques, 16:820, New England Biolabs), UlTma DNA polymerase (from thermophile Thermotoga maritima; Diaz and Sabino, 1998 Braz J. Med. Res, 31:1239; PE Applied Biosystems), Tgo DNA polymerase (from thermococcus gorgonarius, Roche Molecular Biochemicals), E. coli DNA polymerase I (Lecomte and Doubleday, 1983, Polynucleotides Res. 11:7505), T7 DNA polymerase (Nordstrom et al., 1981, J. Biol. Chem. 256:3112), and archaeal DP1I/DP2 DNA polymerase II (Cann et al., 1998, Proc Natl Acad. Sci. USA 95:14250—>5).

While mesophilic polymerases are contemplated by the invention, preferred polymerases are thermophilic. Thermophilic DNA polymerases include, but are not limited to, ThermoSequenase®, 9°Nm™, Therminator™, Taq, Tne, Tma, Pfu, Tfl, Tth, Tli, Stoffel fragment, Vent™ and Deep Vent™ DNA polymerase, KOD DNA polymerase, Tgo, JDF-3, and mutants, variants and derivatives thereof.

Reverse transcriptases useful in the invention include, but are not limited to, reverse transcriptases from HIV, HTLV-1, HTLV-II, FeLV, FIV, SIV, AMV, MMTV, MoMuLV and other retroviruses (see Levin, Cell 88:5-8 (1997); Verma, Biochim Biophys Acta. 473:1-38 (1977); Wu et al., CRC Crit Rev Biochem. 3:289-347(1975)).

B. Exo(−) Polymerases

Methods of the invention provide for an exo(−) polymerase. Useful exo(−) polymerases include wild-type polymerases and mutant or variant polymerases that have a reduced ability to remove incorporated nucleotides from the 3′ end of a nucleic acid polymer, or substantially lack the ability altogether. For example, useful mutant or variant exo(−) polymerases may have about 0.03%, 0.05%, 0.1%, 1%, 5%, 10%, 20%, 50% of the exonuclease activity relative to the counterpart wild-type polymerases. Mutations that reduce or eliminate 3′→5′ exonuclease activity are known in the art and contemplated herein.

Examples of DNA polymerases substantially lacking in 3′→5′ exonuclease activity include, but are not limited to, Taq, Tma(exo(−)), Pfu(exo(−)), Pwo(exo(−)), KOD(exo(−)) and Tth DNA polymerases, and mutants, variants and derivatives thereof.

C. Exonucleases

As discussed above, in addition to a polymerase, methods of the invention provide for a 3′→5′ exonuclease that cleaves bonds, preferably phosphodiester bonds, between nucleotides one at a time from the 3′ end of a polynucleotide. The essential function of the 3′→45′ exonuclease is to recognize and cleave a mismatched base pair terminus.

Enzymes comprising 3′→45′ exonuclease activity according to the invention include, but are not limited to E. coli exonuclease I, E. coli exonuclease III, E. coli recBCD nuclease, mung bean nuclease, and the like (see for example, Kuo, 1994, Ann N Y Acad Sci., 726:223-34). Preferably, the enzyme comprising 3′→45′ exonuclease activity is a DNA polymerase. Useful exo(+) polymerases include, but are not limited to, Pwo DNA polymerase; Vent™ DNA polymerase; Deep Vent™ DNA polymerase; 9°Nm DNA polymerase; UlTma DNA polymerase; Tli DNA polymerase; Pfu DNA polymerase; JDF-3 DNA polymerase; Tgo DNA polymerase; KOD DNA polymerase; and PGB-D DNA polymerase, E. coli DNA polymerase I, T7 DNA polymerase, and Tma DNA polymerase.

D. Polymerase and Exonuclease Combinations

According to the invention, a DNA polymerase is used in conjunction with a second enzyme comprising 3′→45′ exonuclease activity, preferably a DNA polymerase having exonuclease activity. The polymerase and exonuclease are selected based upon a number of different factors. These factors typically include the compatibility of reaction conditions (e.g., pH, buffer composition, temperature requirement, etc.) required by each enzyme. However, in the event that the exonuclease is used subsequently to, rather than simultaneously with the polymerase, each enzyme may have separate reaction conditions specifically suited to the enzyme.

The selection of an exonuclease also may be based upon the desired or required specificity of proofreading activities. For example, one 3′→45′ exonuclease may proofread a G-T mismatch more efficiently than an A-A mismatch, another exonuclease having a different proofreading preference may proofread an A-A mismatch more efficiently than a G-T mismatch.

In general, the relative amounts of exo(−) polymerase and exo(+) polymerase are chosen empirically to achieve a predetermined desired amount of proofreading of misincorporated bases. The ratio of the DNA polymerization activity to 3′→45′ exonuclease activity can be optimized by performing a series of simple experiments in which the relative amounts of the exo(−) polymerase and exo(+) polymerase in the reaction mixture are systematically varied and the synthesis results compared.

According to the invention, the exo(+) polymerase can be present in any amount relative to the first amount, though preferred amounts include about 1% or less, less than about 0.5%, and less than about 0.1% than the amount of the first polymerase. The second polymerase can be present at greater relative amounts, such as about the same or an equimolar amount. In the latter case, the second polymerase can be exposed to the primer/target duplex for a shorter period of time than the first polymerase. In a preferred embodiment, the exo(+) polymerase is present in an amount sufficient to remove incorrectly-incorporated nucleotides that have been added to the primer but insufficient to remove a substantial amount of correctly-incorporated nucleotide.

While the invention is described generally herein in terms of a single polymerase used in conjunction with a single exonuclease, the invention also contemplates the use of one or more polymerases with one or more exonucleases. For example, a polymerase may be used with two different exonucleases in a single reaction; the exonucleases having specificity for different mismatched based pairs. The ratio of total polymerization activity and total exonuclease activity in the combination of enzymes may be optimized for efficiency and fidelity of DNA synthesis.

E. Nucleotides

Nucleotides useful in the invention include any nucleotide or nucleotide analog, whether naturally-occurring or synthetic. For example, preferred nucleotides are adenine, cytosine, guanine, uracil, or thymine bases; xanthine or hypoxanthine, 5-bromouracil, 2-aminopurine, deoxyinosine, or methylated cytosine, such as 5-methylcytosine, and N4-methoxydeoxycytosine. Also included are bases of polynucleotide mimetics, such as methylated nucleic acids, e.g., 2′-O-methRNA, peptide nucleic acids, modified peptide nucleic acids, locked nucleic acids and any other structural moiety that can act substantially like a nucleotide or base, for example, by exhibiting base-complementarity with one or more bases that occur in DNA or RNA and/or being capable of base-complementary incorporation, and includes chain-terminating analogs.

Nucleotides for primer addition according to the invention preferably comprise a detectable label. Labeled nucleotides include any nucleotide that has been modified to include a label that is directly or indirectly detectable. Preferred labels include optically-detectable labels, including fluorescent labels or fluorophores, such as fluorescein, rhodamine, derivatized rhodamine dyes, such as TAMRA, phosphor, polymethadine dye, fluorescent phosphoramidite, Texas Red, green fluorescent protein, acridine, cyanine, cyanine 5 dye, cyanine 3 dye, 5-(2′-aminoethyl)-aminonaphthalene-1-sulfonic acid (EDANS), BODIPY, 120 ALEXA or a derivative or modification of any of the foregoing, and also include such labeling systems as hapten labeling. Accordingly, methods of the invention further provide for exposing the primer/target nucleic acid duplex to a digoxigenin, a fluorescein, an alkaline phosphatase or a peroxidase.

Certain embodiments of the invention are described in the following examples, which are not meant to be limiting.

II. EXAMPLES

### Example 1

### Optimization of Ratio of Polymerase and Exonuclease

Taq DNA polymerase is unable to correct nucleotide misincorporations made during polymerization due to its lack of 3′→45′ exonuclease activity. In general, this would result in the inability of Taq to extend from a newly polymerized strand annealed to a template when an incorrect nucleotide has been incorporated. Pfu DNA polymerase, on the other hand, has an inherent 3′→5′ exonuclease activity and would be able to remove the incorrectly incorporated nucleotide. The polymerase activity of either Pfu or Taq would be able extend from the correctly base paired primer:template. To optimize the efficiency of this process, several ratios of Taq:Pfu DNA polymerase are used in primer extension reactions using 3′ matched and mismatched primers, and the results compared. It would be expected that optimum synthesis product yield would be obtained using a low concentration of Pfu DNA polymerase relative to Taq DNA polymerase.

### Example 2

### Sequencing Using Exo(+)/Exo(−) Polymerases

An optimal ratio of exo(+) Pfu DNA polymerase to exo(−) Taq DNA polymerase is determined as provided in Example 1 such that the Pfu polymerase is present in an amount sufficient to remove incorrectly-incorporated nucleotides that have been added to the primer but insufficient to remove a substantial amount of correctly-incorporated nucleotide. Primer/template duplexes are bound to a solid support in a concentration that provides individually optically resolvable duplexes. The bound duplexes are subjected to serial sequencing-by-synthesis reactions as described in Braslavsky et al., supra. in the presence of one or more labeled nucleotides; and the predetermined relative amounts of Taq and Pfu DNA polymerases. The incorporation of a labeled nucleotide is determined, recorded and the reaction repeated in order to compile that is representative of the complement of the target nucleic acid.

### Example 3

### Exonuclease Activity of Prepared Polymerase Mutants

The 3′→45′ exonuclease activity of wild type Taq polymerase (exo(−)), Tne DNA polymerase (exo(+)), mutant exo(−) Tne polymerase, and mutant exo(+) Taq/Tne hybrid polymerase is measured using a 3′-labeled double stranded DNA. The substrate used is Taq I restriction enzyme digested lambda DNA fragments labeled at the 3′-end with ³HdGTP and ³HdCTP in the presence of E. coli DNA polymerase I. One pmol of the substrate is used in 501 μl reaction containing 20 mM Tris-HCl, pH 8.0, 50 mM KCl, 2 mM MgCl₂, 5 mM dithiothreotol (DTT) with 2.5 units of the different polymerases. The reaction is incubated for one hour at 72° C. The tubes are placed on ice and 101 μl of each reaction is spotted on a PEI plate. Thin layer chromatography is carried out in 2 N HCl. Release of terminal label is measured by liquid scintillation.

It is expected that a negligible amount of labeled nucleotide will be released by the 3′→5′ exonuclease mutant of Tne polymerase and wild type Taq polymerase. However, if full 3′→5′ exonuclease activity of Tne polymerase activity is been retained in the mutant hybrid polymerase, then both 3′→45′ exonuclease proficient Tne polymerase and the Taq/Tne hybrid DNA polymerase should release equal amount of labeled nucleotide.

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein

We claim:

1. A method for sequencing a nucleic acid, the method comprising the steps of:
(a) obtaining a nucleic acid duplex comprising a template and a primer hybridized thereto;
(b) exposing said duplex to a plurality of deoxynucleotide triphosphates, at least one of which is complementary to a nucleotide at a position in said template immediately adjacent to the 3′-terminus of said primer, in the presence of a first polymerase lacking exonuclease activity and a second polymerase having exonuclease activity;
(c) identifying said deoxynucleotide triphosphate that is complementary to said nucleotide; and
(d) repeating steps (b) and (c).

2. The method of claim 1, wherein said second polymerase is present in an amount sufficient to remove misincorporated deoxynucleotide triphosphate at said position.

3. The method of claim 2, wherein said second polymerase is present in an amount insufficient to remove a substantial amount of said complementary deoxynucleotide triphosphate.

4. The method of claim 2, wherein the amount of second polymerase is present as about one percent or less of the amount of first polymerase.

5. The method of claim 4, wherein the amount of second polymerase is present as less than about 0.5% of the amount of first polymerase.

6. The method of claim 4, wherein the amount of second polymerase is present as less than about 0.1% of the amount of first polymerase.

7. The method of claim 1, wherein said exposing step comprises sequentially exposing said duplex to members of said plurality.

8. The method of claim 1, wherein said exposing step comprises simultaneously exposing said duplex to members of said plurality.

9. The method of claim 1, wherein the amount of second polymerase is determined based upon the misincorporation rate of said complementary deoxynucleotide triphosphate.

10. The method of claim 1, wherein the amount of second polymerase is determined based upon the identity of the nucleotide immediately preceding said complementary deoxynucleotide triphosphate.

11. The method of claim 1, further comprising the step of compiling a sequence of deoxynucleotide triphosphates incorporated into said primer.

12. The method of claim 1, wherein said members of said plurality of deoxyribonucleotide triphosphates are detectably labeled.

13. The method. of claim 12, wherein each of said members contains the same detectable label.

14. The method of claim 12, wherein each of said plurality comprises a mixture of adenosine triphosphate, guanidine triphosphate, cytidine triphosphate, and uridine triphosphate.

15. The method of claim 1, wherein said first polymerase and said second polymerase are exposed to said duplex sequentially in separate steps.

16. The method of claim 15, wherein said second polymerase is exposed to said duplex for a shorter period of time than said first polymerase.

17. The method of claim 16 wherein said second polymerase is exposed in equimolar concentration with respect to said first polymerase.
